(19) United States
(12) Patent Application Publication
(10) Pub. No.: US 2004/0248315 A1
(43) Pub. Date: Dec. 9, 2004

(54) Method and device for the analysis of a fluid medium

(76) Inventors: Jens Klein, Heidelberg (DE); STephan Andreas Schunk, Heidelberg (DE)

Correspondence Address: Stephen D Scanlon, Jones Day, North Point, 901 Lakeside Avenue, Cleveland, OH, 44114, US

(21) Appl. No.: 10/488,566

(22) PCT Filed: Sep. 5, 2002
(86) PCT No.: PCT/EP02/09951

(30) Foreign Application Priority Data
Sep. 5, 2001 (DE) 101 43 517.7

(51) Int. Cl. G01N 25/20; C12M 1/00

(52) U.S. Cl. 436/147; 435/283.1

(57) ABSTRACT

The present invention relates to a method as well as a device for analysis of a fluid medium wherein the fluid medium is guided over at least one micro sensor (14) of a sensor assembly, comprising at least two microsensors (14) being identical or different, and wherein the at least one microsensor (14) is thermographically supervised with at least one detector with regard to an amendment of properties wherein the amendment of properties of the at least one microsensor (14) is specific for at least one predetermined component of the fluid medium.

The present invention relates to a method and a device for analysis of a fluid medium in using an array of microsensors wherein the intensity of the heat radiated by the microsensors is preferably detected externally and decoupled, deviated, and evaluated. Therein, the present invention relates in particular for detecting chemical substances in fluid phases.

The main problem of many a sensor technology is the translation of a chemical or physical signal of the sensor into an electrical one. Most often, this is effected by the use of photo-elements (optical electrical), Piezo crystals (mechanical electrical) or semi-conductor cells (thermal orcapacitive electrical).

A very good overview over the current status of the sensor technology is given by WO 00/13004. It is discriminated between chemical sensors, whose principle of deviation is based on the change of electrical properties (e.g. conductivity, resistance or voltage) by contact (e.g. adsorption) of an analyte molecule with the sensor material, and sensors, changing an optical signal into an electrical impulse (e.g. photocell). A further possibility is a chemical-optical “translation” of the signal, such as used in e.g. pH indicators. By a change of the pH value in a chemical environment, an indicator molecule is changing its color or fluorescence properties; these can simply read out visually.

WO 00/13004 relates to an assembly wherein the amendment of the composition of monomer and co-monomers as well as the addition of other functional molecules (e.g. dyes), inserting into the polymer matrix, may develop and optimize materials with sensor and deviation functionalities. The example of inserting dyes as well as the connection with glass-fiber optics describe the development of a sensor assembly for controlling the pH value. A further example highlights the use of conducting polymer composites, which may enable the direct redirecting of the figure. The problems of conventionally developed sensors, however, appear here in the same way since merely a methodic for accelerated optimization of sensor-analyte interactions by parallel multiple production of various materials is described. This does not describe a new sensor technology.

U.S. Pat. No. 4,246,228 describes the probably oldest assembly of a sensor for the detection of combustible gases. A measuring cell with a glowing wire is connected as an element of a so-called Wheatstone bridge. The measuring cell is filled with the analyte gas. If there is a combustion on the wire, the wire's resistance changes, resulting in a current flow of the Wheatstone circuit. The current flow is proportional to the concentration of the combustible gas (e.g. a hydrocarbon) in the measuring cell. This kind of sensor, however, cannot differentiate between hydrocarbons and hydroxide or carbon monoxide; all molecules are integrally detected.

A further development of the above assembly is, e.g. the U.S. Pat. No. 6,109,095. Here, the sensor element is created by a semi-conductive layer of, e.g., ZnO—Fe₂O₃. This layer serves as a selective catalyst for the oxidation of hydrocarbons (H₂ or CO are not oxidized!) and as a “translator” of the chemical signal into an electrical one by a change of conductibility in said layer which can be recorded by means of two electrodes.

U.S. Pat. No. 5,734,091 describes in analogy to U.S. Pat. No. 6,109,095 the design of a nitrogen oxide sensor. The active phase of the sensor is in this case a Bi₂Sr₂YCu₂O_{8+y}. Again, the active mass is exposed as the upper layer with regard to the analyte; the diverting is performed by contacted semi-conductor layers, positioned there-below.

U.S. Pat. No. 5,945,343 describes, as an example, the functionality of a sensor based on the change of the pH value, using an urea sensor: The ammonia forming on the sensor surface when the urea is dissolving (induced by the urease enzyme immobilized on the surface) deprotones an indicator molecule INDH+ to IND. A change of color (fluorescence detection) is associated with this reaction on the indicator molecule, which is then recorded. The most important challenge therein is the stabile immobilization of enzymes as well as indicator molecules. In the present case, this problem is solved by a multi-layer construction of the sensor.

The core point of U.S. Pat. No. 4,874,500 is the production of a microstructured electrode array, wherein the implementation and contacting is effected from, e.g., the backside of a silicon wafer, divided into fields. Each position can be individually addressed and can selectively be used to detect a component (e.g. in human blood) by means of a cover with a specific sensor layer. By a combination of various sensor materials, a complete analysis of an analyte is possible. The task was obviously defined by diminishing the deviation electrodes and constructing a microstructured wafer system.

WO 00/36410 shows the complex construction of sensor assemblies even more clearly, based on localized diverting electrodes. The high number of sensor materials on the array render in particular signal processing and data management difficult. The whole construction cannot be realized but for complex production techniques of microstructuring (etching and masking techniques).

U.S. Pat. No. 5,788,833, too, describes in detail the construction of electronical circuits for diverting the actual signal; the active sensor layers are variable and can be applied according to the problem.

The disadvantage of known technologies is a complex construction, in particular of the sensor carrier (semi-conductor layers), including the contacting and translation of the sensor signal into an electrical signal as well as the data recording. The main problem therein is the diverting of the electrical signal, proportional to the sensor signal.

With regard to the use of infrared-thermographic methods for testing catalysts, the following documents are to be cited: WO 99/34206, WO 97/32208, U.S. Pat. No. 6,063,633 and WO 98/15813. All four documents show the principle of using thermography as a quick analytic method for testing for catalyst activity. The assembly of the instruments, e.g. of WO 99/34206, however, relates to a basically different function of the camera as well as of the choice of materials in the reactor setup than is the case in the present invention. In the present cases, a large number of possible catalysts is merely tested for their activity.

Thus, one problem of the present invention was to provide a method and a device, making it possible to detect chemical substances in fluid mediums more effectively as well as to simplify the constructive expense of a measuring apparatus used therefore, and to simultaneously diminish or avoid high background noise when diverting an electrical sensor signal. Another problem of the present invention is to provide a method for determining performance properties of materials.

Thus, the present invention relates to a method for analyzing a fluid medium, wherein the fluid medium is guided over at least one micro sensor of a sensor assembly, comprising at least two microsensors being identical or different, and wherein the at least one microsensor is thermographically monitored with at least one detector with regard to a change of properties wherein the amendment of properties of the at least one microsensor is specific for at least one predetermined component of the fluid medium.

Moreover, it relates to a device for performing the method in accordance with the present invention, comprising:

(1) means for receiving at least two individual microsensors (14) with or without a sensor carrier (12) with at least two different sections (36), separated from each other, for providing a sensor assembly,

(2) means for letting in and out of at least one fluid medium into at least one section (36) of the sensor device with simultaneous contacting of the fluid medium with at least one microsensor (14),

(3) at least one detector for thermographic determination of at least one amendment of properties of the microsensor (14), as well as

(4) a data processing facility (30) which processes the sections (36) detected when measuring the at least one amendment of properties with the corresponding measuring values corresponding to their position within the sensor carrier (12).

The sections for receiving the at least two microsensors are preferably such sections which are appropriate apertures of arbitrary geometric forms. “Sections” in the framework of the present invention relate to preferably defined locations within the device in accordance with the invention, such as hollow spaces, which are defined due to their coordinates and which are always retraceable. These locations or sections can be appropriate for receiving one or more microsensors.

The sections can be formed byseveral elements of the device, which are e.g. plate- or disk-shaped. Preferably, such a section extends over at least two plates or disks wherein one plate or disk is preferably the bottom (lower part) and the other one the cover (upper part).

A “fluid medium” is in accordance with the invention such a medium which has flowing property proportionate to the expression e^{-ΔE/RT,} wherein ΔE is the energy which has to be overcome in order to allow the medium to flow. This comprises, e.g. fluids, gases, waxes, dispersions, fats, suspensions, liquified materials, powdered solid substances etc. If the medium is liquid, multi-phase liquid systems are comprised as well.

Preferably, a fluid medium is a liquid, particularly a gas-like medium, consisting of one or more components. This fluid medium can, e.g., be the product mixture obtained after the transformation of an educt via catalysts, particularly via a material library comprising at least two potentially catalytic elements.

Within the framework of the present invention, a “predetermined component” is a component of the fluid medium which reacts to one or more, preferably all, micro sensors specifically, analogously to the functionality of a biological system (bionics). Thus, preferably microsensors for all possible components of the fluid medium are contained in a predetermined manner in the sensor device, contained preferably on or in a sensor carrier.

The term “material library” determinates an assembly, comprising at least two preferably up to 10, more preferably up to 100, in particular up to 1000 and more preferably up to 100,000 substances, or (chemical) compounds, mixtures of (chemical) compounds, materials, formulations which are present on/in a substrate in solid, liquid or gasform. Preferred substances in the frame of the present invention, are not gas-like substances such as solid substances, liquids, sols, gels, wax-like substances or substance mixtures, dispersions, emulsions suspensions, particularly preferred solid substances. Therein, in the framework of the present invention, these substances may be molecular and non-molecular chemical compounds or formulations, or mixtures or materials, wherein the term “non-molecular” defines substances which can be continuously optimized or changed, as opposed to “molecular” substances which have a structural expression which can only be changed via a variation of discrete states, i.e. e.g. the variation of a substance pattern.

The substances within the material library may be equal or different, wherein the latter is preferred; in an optimization of test or reaction or process parameters, however, it is also highly possible that the material library contains two or more equal substances or consists of identical substances.

Conventional electronical sensors for detecting substances in the gas phase are generally based, as described initially, on the catalytic composition, chemical reaction or adsorption of substances contained in the gas phase. These substances cause change of enthalpy on the sensor element by the reaction. The sensor elements have to be materials which changetheir specific electrical resistance, depending on the temperature, and which may thus be used for a multitude of materials of the assembly in accordance with the invention. Thus, response signals of the individual sensors may be traced via the Wheatstone bridge. The specific reaction of the sensor to certain chemical substances is achieved via evaluation of the various response signals of the individual sensors. The larger the number of sensors and the more diverse the number of sensor materials, the lesser the danger of transverse sensitivity and of misdetections. Until now, sensor technologies meet a number of limits. For once, all sensor elements have to be electrically contacted. The methods now available for contacting thus set the degree of miniaturization of the sensors. Additionally, an electronical contacting is susceptible to noise fields, such as strong magnetic fields etc. and can only be operated within a temperature window which is most often highly limited.

The method in accordance with the invention compensates for these disadvantages. Since no electrical contacts to the sensor elements are required, there is no principle limitation to miniaturization. The range of temperatures in which the sensor element can be operated, is significantly larger. Neither the sensor materials nor the carrier have to be semi-conductive.

In particular the aspect of miniaturization due to the lacking electrical contacting leads to entirely new perspectives with regard to the sensorial potential of the material. Sensor elements can be provided on a little number of square centimeters as in animals' organs of smelling. This makes differentiating between chemical substances possible which cannot be achieved by means of conventional sensor technology. The danger of transverse sensitivity is limited by a large number of diverse sensors as in the biological system; even molecules which are chemically very similar, can be discriminated and even substance mixtures can be identified and quantified clearly.

The analogy to biological systems is obviously given in the system in accordance with the invention. Exactly as in an animal sensing organs of smell or taste, millions of different sensors (differentiated sense cells) detect the applied sample wherein all sensors show an individual response to specific substances. This does not mean that sensors merely react to one substance but that each substance creates with each sensor a specific signal in specific strength, i.e. the signal intensity is proportional to the concentration of a substance or a mixture of substances. As in the biological system, transverse sensitivities of the sensors are not excluded. Sensor 1 can create the same signal with substance A as sensor 2 with substance A. Differentiating between substances and quantifying them is not made possible but for the plurality, diversity and multitude of all sensors as well as the sensorial signals' strength. Therein, the method functions similarly to the biological systems (bionics): the contact of sensor on substance creates a specific reaction on every microsensor/sensor material. The sum of all reactions results in a complex pattern which is specific for a substance or a substance mixture. Therein, the entire patterns are evaluated in biological systems and the identification of a smell and/or taste is quasi performed as a recognition of patterns. The sum of all receptors of a system of smell and/or taste enables redundancy to occur with regard to the sensorial signals. The performance of pattern recognition is thus for the most part the transformation of the complex sensor signals into information of individual substances or substance mixtures. A further challenge in biological systems is the continuous renewing of the smell sense-organs: From the viewpoint of the sensors, there is a steady change of pattern. The evaluation system has to adapt flexibly to the changed neuron population.

The method in accordance with the invention relies on paths which lean against these biological solutions and provide technical teachings for their solution. Thus, the evaluation of the signals of all sensors is performed by means of the infrared detector in the sense of a recognition of patterns, wherein the contained sensor pattern is compared with a pattern data bank. The pattern data bank can therein be open in order to receive new patterns steadily. In order to avoid a misinterpretation in critical analyses, the sensor may be highly redundant, i.e. special sensors are not different from one another. This redundancy intrinsic to the system prevents a sensor breakdown.

The microsensors may also be subjected to a regeneration (in the biological context, this would mean a relaxation without olfactory stimulant or taste stimulant) in order to make a measuring under standard conditions possible. Comparing with reference substances can also lead to an increased robustness of the system. The dosing of reference substances can, on the one hand, clearly ensure the identity and/or the concentration of substances or substance mixtures, on the other hand, a change of sensor properties can easily be detected. This is entirely analogous to biology. Changed sensor properties can either result in an exchange of the sensor element or a new calibration of the sensor elements is performed in saving the new sensor responses.

Further-reaching software algorithms may also be used, by means of said algorithms the response of a certain sensor to a certain substance or substance mixture may be predicted; appropriate algorithms may be e.g. neuronal nets or evolutionary algorithms.

Thus, these systems can be called bionic or pseudo-bionic. Bionics is the imitation of natural biological function principles in techniques. The whole procedure can thus be called a bionic or pseudo-bionic or quasi-bionic sensography.

The term “sensor assembly” relates to an assembly comprising at least two, preferably up to 10, further preferably up to 100, in particular up to 1000 and further preferably up to 108 microsensors. This assembly can be provided on or in a sensor carrier. In another embodiment, the sensor assembly is comprised of a number of microsensors, forming a solid bond without the help of a sensor carrier, e.g. if the microsensors are wire- or roll-shaped.

The term “microsensor” relates to non-gaseous substances, such as solid substances, oxides, salts, liquids, sols, gels, wax-like substances or substance mixture, dispersions, emulsions, and suspensions, cells, anti-bodies, enzymes, bacteria, proteins, proteides, fungus, viruses, priones, DNA and RNA, particularly preferable solid substances. Therein and in the framework of the present invention, the microsensors used may be molecular or non-molecular chemical compounds or formulations or mixtures or materials, wherein the term “non-molecular” defines microsensors which may be continuously optimized or changed, as opposed to “molecular” microsensors, the structural formation of which may merely be changed by a variation of discrete states, e.g. the variation of a substitution pattern. The microsensors may be equal or different, wherein the latter is preferred. However, it is also possible that the sensor device comprises two or more equal microsensors or consist exclusively of identical microsensors.

Microsensors may be non-porous or porous (macro-, meso-, or microporous) and may be present in general in every geometric form, e.g. as films or monolayers, preferably as three-dimensional bodies.

All production methods known to the person skilled in the art are possible as production methods for microsensors. As examples be mentioned: sputter methods, coating methods, CVD-, PVD-methods, electrochemical methods, impregnating methods, precipitation methods, spray methods, etc.

Moreover, microsensors with continuos and/or discontinuous composition of the sensor material are to be discriminated. Therein a microsensor with a discontinuous composition has a discrete variation of sensor materials and a microsensor with continuous composition has a continuous variation of sensor materials.

At least the lower limit for the size of microsensors with discontinuous composition is oriented on the maximum definition of the detector. The upper limit with regard to the microsensors' size is generally not limited, however, it is regularly oriented on the maximum field of view of the detector. In order to enlarge the field of view, more than one detector may be used, wherein the fields of view of the individual detectors may be provided adjacently or overlappingly.

Microsensors with continues composition are typically not limited in their size. The microsensors, however, have a design such that the change of properties of the microsensors is still detectable in a sensible way within the frame of the measuring method when contacting the fluid medium.

Preferably, the spacing between the microsensors is also merely limited by the definition of the infrared camera, preferably used as detector.

In an alternative embodiment, the microsensor may also be comprised of a carrier body and a sensor material applied thereon continuously and/or discontinuously. Such carrier bodies may principally be all two- or three-dimensional devices and bodies with a rigid or semi-rigid surface, which may be flat as well as provided with apertures, pores or borings or channels. The carrier body has to be suited for receiving the sensor material(s). With regard to the outer form of the carrier body, there are no limitation as long as the device is two- or three-dimensional or the body is two- or three-dimensional. Thus, the carrier body may have the form of a sheet-like product, e.g. a foil, of a wire-like formation, of a fabric, a mesh, and a knitted and/or crocheted ?, of a ball or hollow ball, of an ellipsoid boy, of a cuboid, of a cube, of a cylinder, of a prism, or of a tetrahedron.

Preferably, however, microsensors without carrier bodies are used which are provided within the respective sections of the sensor carrier and which can have generally the same shape as the carrier bodies described above. Wire-shaped microsensors may e.g. be assembled in the shape of a woven material of the wires to a “carrier-free” sensor assembly.

The size of the area is generally not limited in the case of continuous microsensors which are e.g. applied as an element gradient on a sensor carrier in a sheet-like form. Preferably it ranges from 1 μm² to a range of square meters.

For microsensors with continues composition, the afore mentioned definition of spacings is to be limited in such a way that, e.g., an element gradient which can be limited via its surface is a section on the sensor carrier or sections with predetermined concentrations may be defined within the gradient.

The present invention further relates to a sensor device, comprising at least two microsensors wherein the microsensors' composition changes continuously or discontinuously.

The term “sensor carrier” as a preferred means for receiving at least two individual microsensors principally comprises all devices with a rigid or semi-rigid surface, such as plates, wires, materials woven from wires, balls, hollow balls, cubes, honeycombs, etc. which may be planar or provided with recesses or borings or channels, respectively. The sensor carrier has to be appropriate for physically separate the at least two individual microsensors into at least two different sections, separated from each other. The microsensors may be provided in the sensor carrier in one, two, or three dimensions, i.e. adjacent to each other and stacked upon each other in different planes.

Preferably, the means for receiving the at least two individual microsensors and/or the at least two individual microsensors are arbitrarily exchangeable. The sensor carrier can therein be e.g. an exchangeable sandwich unit, wherein the microsensors are arranged between two silicon wafers and wherein a microstructure, e.g. in the form of channels, is provided for distributing and conducting the fluid medium.

Alternately, the sensor carrier may also be non-unporous, preferably planar, wherein the microsensors are overflown by the fluid medium. In this case, there is no flow-through of the sensor carrier. Combinations of porous sensor carrier and non-porous microsensor and vice versa as well of porous sensor carrier and porous microsensor as well as of non-porous sensor carrier and non-porous microsensor are also possible.

Preferably, the sensor carrier comprises channels, which are parallely passing through, and may comprise a wire net or a foam ceramics, among others.

Therein and further preferably, it can be made similarly to an integrated material chip or have a construction like the analysis and receiving area with membranes, preferably pore membranes, described in DE 101 17 275.3.

The geometric arrangement of the individual sections to each other can therein be chosen arbitrarily. E.g. the sections may be arranged in the form of a line (quasi one-dimensional), a checker board pattern or like a honeycomb (quasi two-dimensional). In the case of a sensor carrier with a multitude of channels, passing through in parallel, the arrangement becomes obvious when a cross sectional surface is viewed vertically to the channels' longitudinal axis: a plane results wherein the individual channel cross-sections are rendering the various spaced sections. The sections may also be provided in a thick packing—e.g. with channels with circular cross-section—so that different rows of sections are misaligned with respect to one another.

The sensor carrier comprises a multitude (at least 2) of “sections”. Preferably, these sections are areas of the channels, however, they can also represent individual physically spaced sections of a planar sensor carrier or a carrier provided with recesses, e.g. in the form of a titation plate. The channels preferably connect two surface areas of the sensor carrier and pass through the sensor carrier. Preferably the channels are arranged in parallel. The sensor carrier can therein be made of one or more materials and may be massive or hollow. It may have every appropriate geometrical shape. Preferably, it has two parallel surfaces, wherein there is each one opening of the channels. The channels are therein provided vertically to these surfaces. An example for such a sensor carrier is a cuboid or cylinder wherein the channels run between two parallel surfaces. However, a multitude of similar geometries is possible, in particular also with horizontal channels.

The invention in accordance with the present invention is preferably provided with channels as means for guiding in and out of the at least one fluid medium within the sensor carrier, wherein the channels may also connect a multitude of sections.

The term “channel” describes a connection passing through the sensor carrier of two openings provided on the body surface, preferably being provided as means for guiding in and out a fluid medium into and from the sensor carrier, wherein a part of the channel, preferably with an enlarged cross section, serves as a section for receiving at least one microsensor. The channel can therein have an arbitrary geometry. It can be provided with a cross sectional surface changeable over the length of the channel or preferably a constant channel cross-section. The channel cross-section can, e.g., have an oval, round or polygonal contour with straight or bent connections between the corners of the polygon. A round or equilateral polygonal cross section is preferred. Preferably, all channels within the sensor carrier have the same geometry (cross section and length) and are in parallel.

A sensor carrier of a massive material which can in turn be constructed from one or more initial materials, is preferably provided with channels. The channels' geometry can therein be arbitrarily chosen as explained above for the channels in general. The channels can e.g. be left open while forming the massive body/block, e.g. by extrusion of an organic and/or inorganic form mass (e.g. by a corresponding nozzle geometry in extrusion). Preferably, the sensor carrier is made of one or more metals. The channels can be provided for example, in the sensor carrier by lithographic methods, etching methods, LIG methods, laser ablation methods, boring methods, milling methods, eroding methods, lapping methods (e.g. ultrasonic lapping), ECM methods, screen processes, lithography galvano casting, embossing methods, punching methods, etc.

The assignment of the microsensors to the individual sections is therein preferably predetermined.

The term “predetermined” means that e.g. a row of different or identical microsensors, e.g. adsorbents or catalyst or catalytic precursors, are applied into the sensor carrier in such a way that the assigning of the respective microsensors, e.g. catalysts or catalytic precursors, to the individual sections is recorded and is later recalled e.g. in the evaluation of the analysis of the change of properties, preferably for selectivity determination of e.g. catalysts, in order to make possible a clear assigning for certain measuring values to certain catalysts.

The production and distribution of the microsensors on the different sections of the sensor carriers is preferably controlled by a computer, wherein the respective composition o a microsensor and the position of the section within the sensor carrier is stored in the computer and may be polled later. The term “predetermined” thus serves for differentiating between an arbitrary or statistic distribution of the individual microsensors to the sections of the sensor carrier, which is also possible in an alternative embodiment.

In accordance with the invention, the change of properties of the at least one microsensor is measured by at least one optical and/or thermographic detector, wherein an infrared camera or an assembly of infrared diodes or a displaceable infrared diode is used preferably as a detector. The detection can also be done by means of specially configured optical near-field microscopy.

Generally, all detectors known to the person skilled in the art can be used which are appropriate for detecting infrared radiation. As examples be mentioned: pyroelectric vidicon, bolometer arrays and IR quantum receivers or a detection by means of Schlieren methods.

During detection, the detector reacts preferably directly or indirectly to a change of temperature or an input of energy. The detection is performed preferably without contact; thus it is preferably a temperature measuring without contact.

In an alternative embodiment, the detection of the infrared radiation can also be performed via an intermediate detector.

A “Change of properties” in the framework of the present invention is preferably to be understood as a thermal change of properties. The microsensor becomes therein preferably hotter, colder or can have the same temperature as a certain reference material or changes its emissivity with regard to a reference material. Reference materials may be equal or different microsensors or sensor carriers. Generally, temperature and/or emissivity differences are thus preferably measured. The change of properties of the method in accordance with the present invention can be caused by: thermoelectric effects, Peltier effects, Chelat effects, adsorption such as chemisorption and physisorption, desorption, catalytic reactions, complex formation and molecular recognition. Preferably, the change of properties can be reversed.

The thermographic detector preferably used for the measuring of change of properties is preferably an infrared cameras such as an AIM/Aegais PtSi 256×256, which operates in accordance with the principle of infrared thermography, wherein caloric changes as well as the microsensors' emissivity behavior is preferably measured.

The “thermographic supervision” is preferably effected by means of infrared thermography. This is a method for making visible and recording temperature distributions and changes on surfaces of objects by means of heat radiating off the object. A heat picture (thermogram) is obtained wherein the different colors or gray-scale values are translated into an electrical voltage signal by means of a transformation of the long-wave infrared radiation. This matrix of voltage values can e.g. be visualized as false-color photos and/or be further processed directly. Heat pictures can be created by means of photographic recordings with infrared-sensitive photo material or by means of heat-picture devices. In these devices, the heat radiation given off by the object is conducted to an infrared detector (e.g. indium antimonide, cooled by means of liquid nitrogen, in thermovision cameras) via an infrared optic and/or an optomechanical scanning mechanism (e.g. rotating mirror polygon and tilting mirror). The radiation of the “thermal scene”, divided into individual point elements thus hits the detector in a predetermined sequence, freeing an electron without delay for every incident radiation quantum. The amplitude of the electrical output signal is proportional to the radiation performance which is used for brightness or color control of a TV monitor whereon the thermogram becomes visible. Heat displays, operating with a pyroelectrical vidicon as infrared sensor or with CCD infrared detectors for directly viewing and examining do not require a complex mechanical scanning system.

The term “thermographic” is not limited to infrared thermography. Optical measuring methods or detectors for measuring the change of properties in connection with e.g. color or lengths changes, volume changes or deformations can be taken into consideration when measuring the change of properties. Interferometric and pyro-technical measuring methods are also possible.

Moreover, the optical and/or thermographic detector detects in the method according to the invention the heat intensity given off by the at least one microsensor, e.g. in a detector measuring range from 3 to 5 μm. In order to achieve reliable and particularly comparable measuring results, it is also possible to keep the flow density as well as the flow speed of the fluid medium constant in every measuring and of course also during measuring.

A central point of the method according to the present invention is the fact that the signal transfer from the at least one microsensor to the at least one detector is performed without an electrically conducting connection, in particular wireless. The deviation of the signal by means of a wireless external signal has in particular the advantage that it is possible to measure outside of the surroundings in the case of e.g. explosive surroundings and that the system is less dependent on temperature and/or corrosion. Another advantage is the possibility to easily exchange the microsensor or sensor material due to the modular construction.

The change of properties in the connection with a heat emission is therein externally and optically detected, preferably by means of an infrared camera, and the information (e.g. data points, control commands, time indications) are processes in the form of data preferably by means of a data processing facility. “Processing” can be, e.g., a transformation, processing, evaluation, storage, etc. of the data. Thus, there is an indirectly optical evaluation of a thermal information.

In this assembly, the method in accordance with the invention can e.g. be used for determining the performance properties and the selectivity of materials, in particular of catalysts, by means of an infrared camera.

Further possible applications of the method and the device in accordance with the inventive method and the inventive apparatus are e.g. detection of drugs, samples, explosives or other substances, e.g. in the luggage of passengers, in particular in air traffic, detection and identification of warfare agents, pesticides, environmental poisons, use in the process control, the quality control, e.g. in the food, animal food and cosmetics industry, in the area of forensics (forensic detection), in the securing of evidence, for chemical analysis with or without coupling with already-known analyzing methods, for olfactory tests in the consumer goods industry, for detecting of certain mineral resources such as oil, gas, methane hydrate; in the extra-terristic research.

The method in accordance with the invention thus makes possible to qualitatively and quantitatively characterize a fluid or fluid mixture. Thus, the possibility exists for the method to be used as a detector system for other analysis and separation methods. Coupling or integral solutions are possible, wherein the method in accordance with the invention is preceding or following. Preferred is the use as detector system for chromatographic methods such as GC, LC, HPLC, DC; GPC, SEC; SFC; as well as other separation methods known to the person skilled in the art. After separation and/or partial separation of the fluid mixture, individual substances, substance mixtures and unseparated fluid samples are analyzed and characterized by means of the method in accordance with the invention.

Separation is the complete resolution of a fluid or substance mixture into the individual substances. Partial separation is the merely partial resolution of the substance mixture into partial mixtures.

In another embodiment, the analysis or separation method, preceding or following, can also be used as purification or conditioning step of the fluid sample. Possible is e.g.: separation and/or partial separation of substances with a transverse sensitivity, which would result in a similar pattern (“fingerprint”) with similar intensity on a sensor assembly, separation and/or partial separation of substances which might poison, dissolve or influence in any other way all or individual sensor materials, derivatization of substances which then result in an unambiguous pattern on the sensor assembly, derivatization of substances in order to increase or decrease the intensity on a sensor assembly, etc.

A separation and/or partial separation of fluid mixtures can, however, also be achieved by correspondingly porous or surface-treated or polar/unpolar layers or bodies within the sensor assembly.

By means of differing heating or cooling zones on the sensor assembly, it is also possible to adsorb fluids selectively at first in e.g. porous microsensors and to free them after a time t by means of an increase of temperature in this section in order to then identify them via the pattern (the fingerprint). The separation or adsorption may be achieved by the following effects: polarity, acidity, basicity, chemisorption, physisorption, molecular size, electrical charge of the molecule.

“Performance properties” are preferably measurable properties of materials or substances, e.g. of a material or material library which can be evaluated within an automatic testing (analysis) by means of the method in accordance of the invention. The term “performance properties” is described in DE-A 100 59 890.0 in more detail; thereto, it is referred.

Thus, the at least one detector in the form of an infrared camera is also part of the device in accordance with the invention.

Therein, the optical field of view of the camera may be decisive for the size of the sensor assembly wherein every individual microsensor can also be evaluated individually. It is also possible, that the infrared camera merely scans individual sections and that the recorded pictures or matrixes are combined to a large picture if the infrared camera is arranged in a movable. Of course, the infrared camera can also analyze for changes of properties merely individual sections of the sensor assembly, i.e. not the whole sensor assembly. Thus, it is easily possible to use the sensor device with various temperatures in order to guarantee an optimal operation temperature of all micro sensors.

By means of a channel system adaptable to the respective problem, it is possible to sensibly combine various microsensors (i.e. sensor materials with selectivities for various molecules to be quantitated, preferably in a gas phase). The individual sensor materials can therein be synthesized e.g. via the synthesis for inorganic materials described in DE 10059890.0. Certain known instrumental arrangements (e.g. the combination of infrared thermography and mass spectrometry) can be used for the specific proof of target molecules to test the applicability of certain material combinations. Appropriate materials are then sorted out and used as microsensors of a sensor assembly.

The arrangement of the microsensor in the sensor device as well as the connection of the microsensors by means of a channel system, which can be chosen arbitrarily, solely defines the sequence with which a flow of a fluid medium to be analyzed, e.g. a waste-gas flow, flows from a reactor unit over the individual microsensors with corresponding quantification selectivities for different product molecules. It is also possible to sequentially conduct waste gas from a multiple reactor over a sensor device in using a multiport valve. Therein, a microsensor with a different selectivity is provided in every channel of the array, i.e. the whole waste-gas is examined for several target molecules. One microsensor e.g. responds selectively to aromatic aldehydes, a second to aromatic alcohols, another one to aromatic acids, another one to aromatic hydrocarbons, etc.

The device in accordance with the invention further preferably comprises a casing wherein the sensor carrier is provided. Preferably within this casing, the device moreover comprises means for heating and/or means for cooling the casing and/or the sensor carrier.

The means for heating and/or cooling the casing and/or the sensor carrier can preferably be controlled or regulated or set with regard to the temperature individually. The heating and/or cooling elements are preferably arranged in such a way that a predetermined temperature profile can be created for the whole sensor carrier.

In one embodiment, the means for heating and/or cooling comprise electrical heating elements, such as e.g. welded resistance wires, heating spirals or also heating cartridges. Alternatively or additionally, the means for heating and/or cooling may comprise channels which are provided with heat-carrier materials such as gases, liquids, solutions or melted materials.

The temperature regulation can also be adjusted to the individual sections of the sensor carrier also in accordance with another point of view of the invention. Therein, at least two meandering heating or cooling elements, provided in an angle of 0 degree, can be used wherein the angle preferably amounts to 90 degree. Further embodiments with a plurality of individual heating spirals or heating cartridges, which are provided in the spiral-shaped, concentrically or zigzag-shaped, are also possible.

The sensor carrier with the microsensors arranged therein is heated or cooled appropriately by means of these heating or cooling elements. With regard to the embodiment of the heating and/or cooling device, there are no limitations as long as the element is appropriate for sufficiently heat or cool the sensor carrier. An arrangement of channels purged with heated or cooled fluid would also be possible. An active heating or cooling of the casing with sensor carrier by means of externally provided heating and/or cooling means may be used alternatively to or in combination with the aforementioned embodiment.

In another embodiment of the device in accordance with the invention, the casing has an infrared-transparent window wherein the infrared detector is preferably provided outside the casing, preferably in front of the infrared-transparent window.

In particular in order to be able to achieve an even more flexible arrangement and field of view of the infrared camera, the casing and/or the sensor carrier may be comprised entirely of silicon and/or sapphire or another appropriate infrared-transparent material.

Sensor carriers which are also appropriate, may also consist of shale or ceramics with the properties of a black radiator or body. An emissivity correction may optinally be performed in accordance with a method known to the person skilled in the art, e.g. in accordance with the thermal differential method as described in WO 99/34206.

In another preferred embodiment, the device in accordance with the present invention furthermore comprises at least one multiport valve and/or a device for equal distribution of the fluid medium over the at least two microsensors, preferably via all microsensors. Thus, letting in various fluid mediums into the sensor carrier can be controlled e.g. as a sequential admission to selected microsensors.

Individual microsensors, too may be connected to one or more sequential gas admissions in order to, for example, regenerate or calibrate the microsensors.

Devices for equal distribution or for distribution in a defined homogenous or defined inhomogeneous way of the fluid medium via the at least two microsensors, preferably via all microsensors, may be e.g. porous membranes, radial and/or centric gas inlet ports or combinations thereof, restrictions, capillaries, all of them preferably in connection with suctions, wherein the corresponding elements are to be preferably arranged radially and/or centrally.

As already mentioned above, the most important advantage of the present invention is the redundancy of complex contacting and diverting of the electrical signal proportional to the actual sensor signal. Thus, a large number of microsensors (corresponding to the size of the camera's field of view) is also easy to handle. The high temperature resolution, achievable in infrared thermography cameras by means of a corresponding instrumental and material set-up, also avoids the problem of high background noise in electrical signal processing, which results by the actual signal being recorded indirectly, i.e. by e.g. adsorption phenomena on an chemical sensor layer, the electronical surroundings of this layer change which also influences the electrical properties of the contacted semi-conductor layer, provided there-below indirectly. Even when a semi-conducting, directly contacted sensor layer is used, the electrical conductibility or its resistance is substantially controlled by other influences such as e.g. temperature, wherein the actual sensorial phenomenon is merely secondarily changing these material properties.

When indirectly detecting the sensor signal, as in the case of the present invention, all other influences can be excluded by correction. Furthermore, a complex control and evaluation unit for the various sensors becomes redundant, the evaluation is performed variably by means of software tools, assigning the intensities of the infrared array detector easily to the respective materials of the microsensors. Calibrating can be performed via the whole sensor arrangement simultaneously by means of corresponding calibration gas mixtures; the software then allows for an automatic assignment of the intensity (concentration). Furthermore, the openness of the system in accordance with the present invention makes the use of further analysis methods possible in a preferred embodiment. e.g. another integral analysis such as e.g. the use of a CCD camera for recording color changes by e.g. means of covalently bound indicator molecules (e.g. pH indicators for proving basic or acidic molecules) might be possible. On the other hand, the subsequent analysis of the waste-gas by means of MS is possible.

Another advantage is the substantially easier synthesis of the materials for the microsensors. In the conventional proceeding, the range of possible synthesis methods is very limited due to the complex carrier (semi-conductor carrier with electrode contacts), precipitation of the gas or liquid phases are preferably used. The lack of control of the material morphology implies a worse signal/noise ratio.

In the case of the present invention, the carrier of the microsensors does not have any further function; thus, there is a multitude of synthesis techniques. The production of bulk materials is also possible, preferably, however, the method as per DE 100059890.0 is used since many different materials can be tested for their being appropriate in the respective analytical problem in this way and are then used in a sensor device specifically assigned to the problem when answered in the positive. By means of the synthesis of sensor materials, e.g. on small ceramics balls as carriers, it is easily possible to produce a large amount of materials so that the material can be easily exchanged. The exchange of an individual sensor on its position within the array is easy since the rest of the construction is not influenced thereby. Nothing has to be changed on the signal deviation (infrared camera).

Moreover, the device in accordance with the invention is variably usable since the easy exchange of the sensor device guarantees for a completely different functionality. The infrared camera remains in its position (preferably above the sensor device) wherein the change of the reactor unit for the sensor arrangement makes the whole construction usable in a plurality of catalytic and/or other reactions. Thus, the present construction in its entity is financially more advantageous then the new construction of e.g. a sensor device with electronical signal deviation.

The method in accordance with the invention, which any also be called “infrared sensography” due to its combination of sensor device and infrared thermography is not limited to the micro field but can also be used in large-scale facilitates or in the motor vehicle techniques assuming that the sensors or the sensor device are correspondingly dimensioned/scaled.

The microsensors or the method in accordance with the invention or the device in accordance with the invention can furthermore be used for controlling chemical reactions.

Moreover, the present invention relates to a computer program with program code means for performing the method in accordance with the invention or for controlling and/or regulating the device in accordance with the invention, and a data carrier with the computer program.

Moreover, the present invention also relates to the development and optimization for sensor materials which can be used as microsensors. The method in accordance with the invention can thus, in one preferred embodiment, also be used for finding or optimizing a microsensor or appropriate sensor material to solve a given analytic problem. The result of this embodiment are one or more microsensors which can then be used in common devices with common electronical contacting. The advantage of this embodiment is the quick optimization of e.g. one individual microsensor or sensor material to the question posed; individual materials can always be compared with the known pattern of a substance via a sensor device and be optimized. If the individual material's behavior is analogous to the sensor device with regard to selectivity and intensity, the sensor material for a substance has been found.

In detail, the following method is suited for developing microsensors/sensor materials: in a first step, a plurality of sensor materials with large diversity is contacted with a fluid flow of the substance to be analyzed (target substance) alone or in mixture with others (transverse sensitivity, selectivity). The pattern (qualitative analysis) as well as the intensity (quantitative analysis) of the pattern are processed as number matrix (table of voltage values) or as false color photo. Within this first plurality of sensor materials, those are chosen which have a selective behavior to the change of substances (with or without target substance) or to the concentration change of the target substance. Within this parameter range of the synthesis of the chosen materials, further parameters are varied in a second step in the next synthesis or the microsensor properties are changed by adding further substances. The sensor device with the new, changed microsensors is again contacted with the target molecule and the response as well as the intensity of the signal is recorded and processed, etc. By iteration of the steps 1 and 2, only one material or few materials may remain at the end, which have the desired properties regarding the target molecule and can be used as selective microsensor for this substance.

Therein the term “synthesis parameter” relates to the totality of parameters, describing the production and/or testing of potential sensor materials for a microsensor within the frame of individual steps or the totality of the method in accordance with the invention, wherein preferably merely the strictly mathematical or scalar definition of a sum of non-redundant vectors is used for the description of the test and/or reaction and/or production parameters, i.e. the parameter range can also include redundant vectors or scalars.

In this way, whole sensor devices for analysis problems can be produced. It is also possible to optimize sensor devices in the form of the arrangements on which this method is based; e.g. the overdetermination (redundancy) of the sensor devices can be minimized or the diversity of the individual microsensors can be adapted to the analysis problem.

By means of the method in accordance with the invention, the production times for microsensors can be considerably shortened as well as selective microsensors can be found for complex analysis problems within acceptable periods of time. The expense for an electronic contacting as well as for micromechanical techniques for diverting the sensor signal for the totality of all microsensors is redundant during development. An optimized material can be provided with electrical contacts analogously to conventional sensor technology not until it has been found.

Thus, the present invention also relates a method as in question here, wherein the evaluation of the changes of properties found by the thermographic supervision is performed by means of a pattern recognition of the way which compares the found measuring results with regard to property changes to pattern data bases, and that, preferably automatically, identity and quantity of at least one component of the fluid medium is determined thereof.

Moreover, the method in accordance with the present invention is also appropriate for developing and finding new sensor materials, preferably in the way described above.

Some preferred embodiments are described in the following in accordance with the accompanying drawings. Therein are:

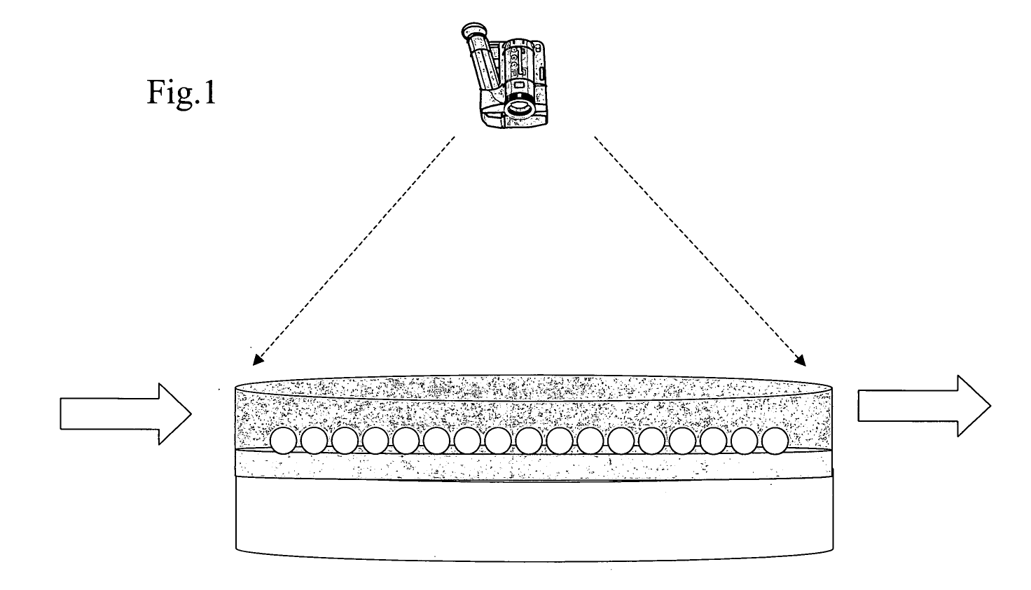

FIG. 1 a schematic view of the device of the invention;

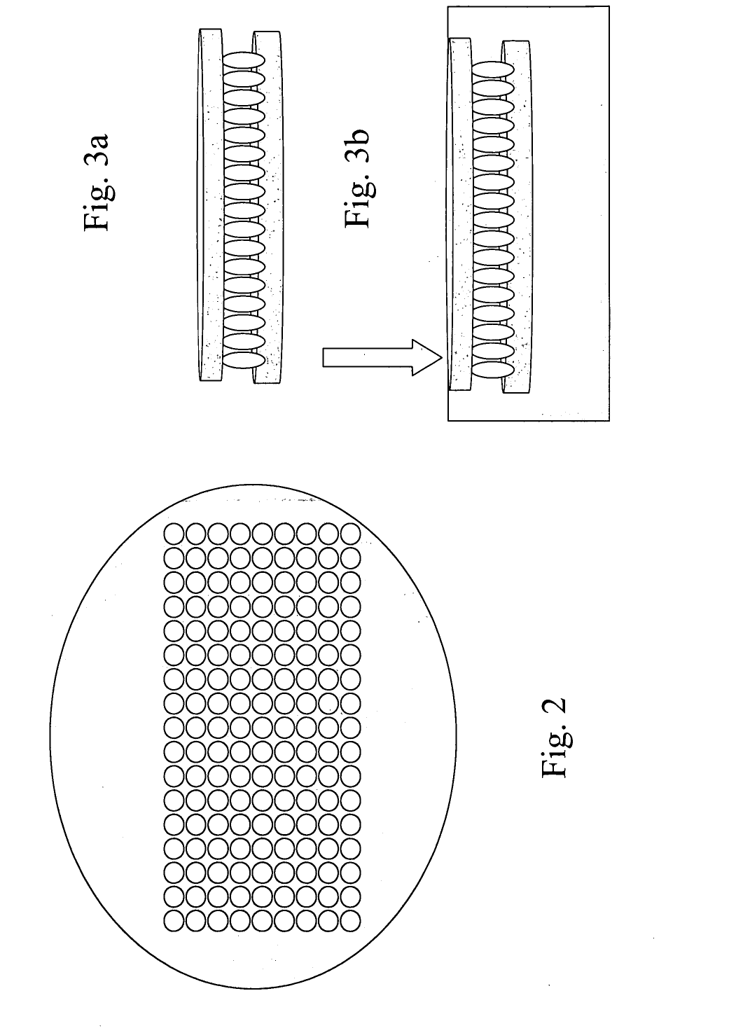

FIG. 2 a sensor carrier with sensor device consisting of microsensors;

FIG. 3a an exchangeable sensor carrier in the form of a sandwich unit,

FIG. 3b a sensor carrier of FIG. 3a with heating element;

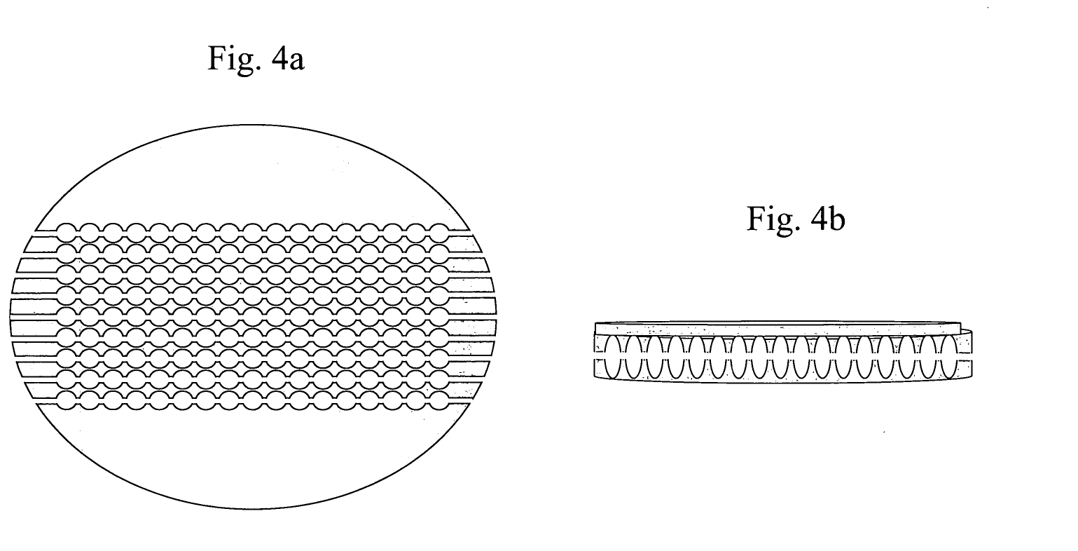

FIG. 4a a sensor carrier with parallel sequencing of microsensors in each row;

FIG. 4b a side view of the sensor carrier of FIG. 4a

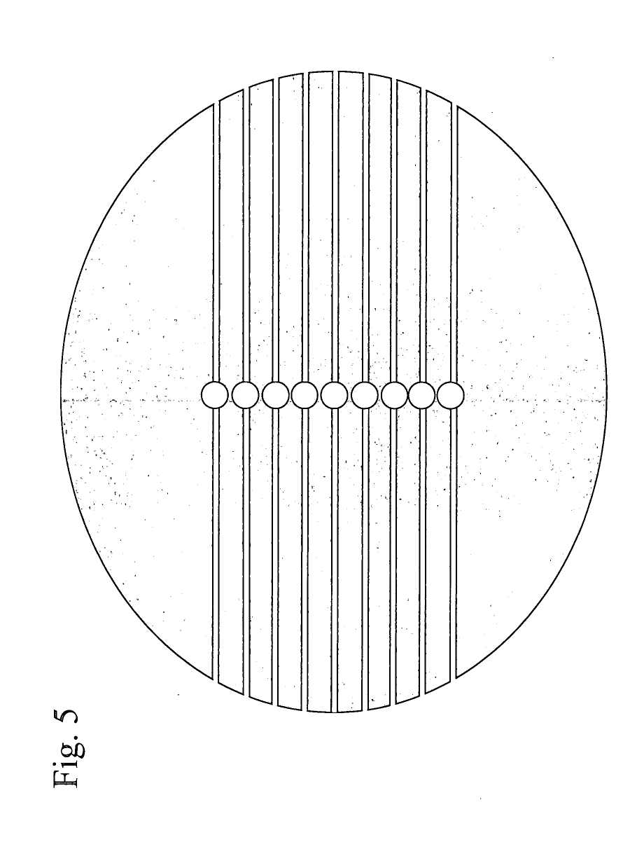

FIG. 5 a sensor carrier with linear sensor arrangement

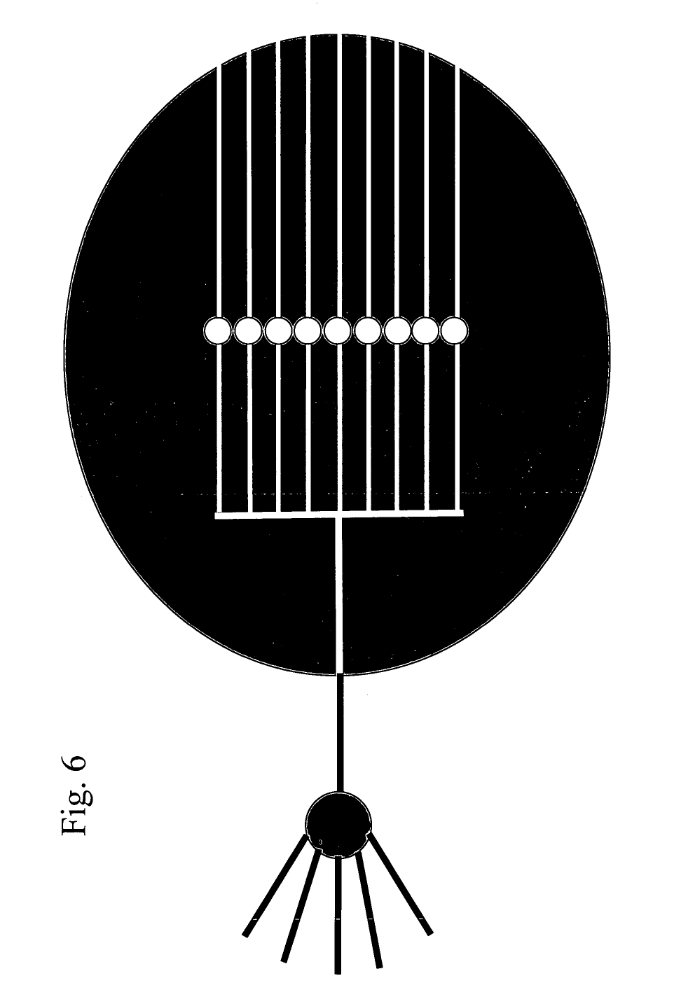

FIG. 6 a sensor carrier with integrated multiport valve

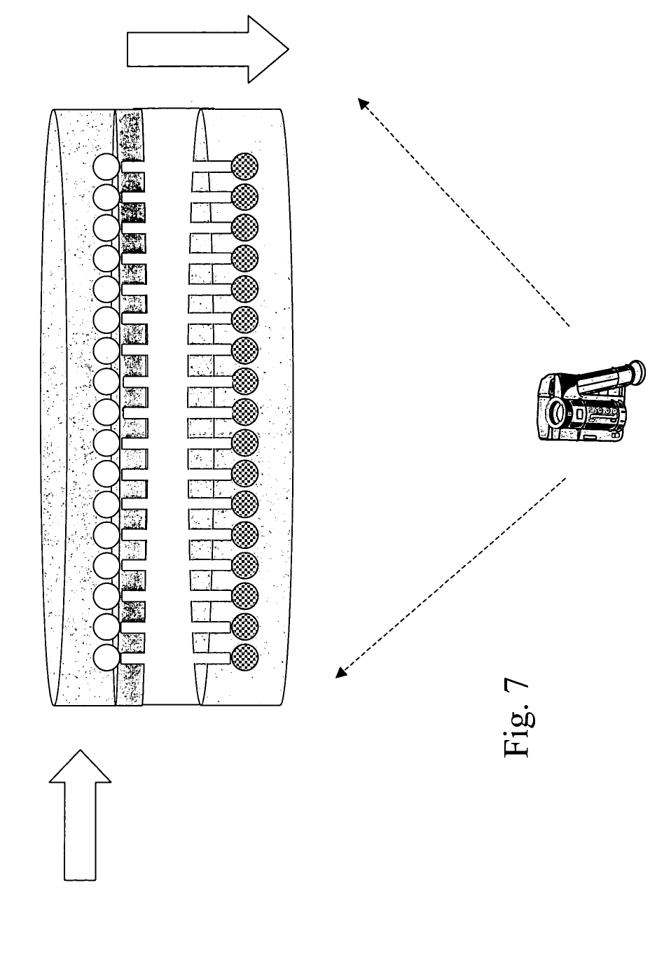

FIG. 7 a possible arrangement of the inventive device as analysis system for a test reactor

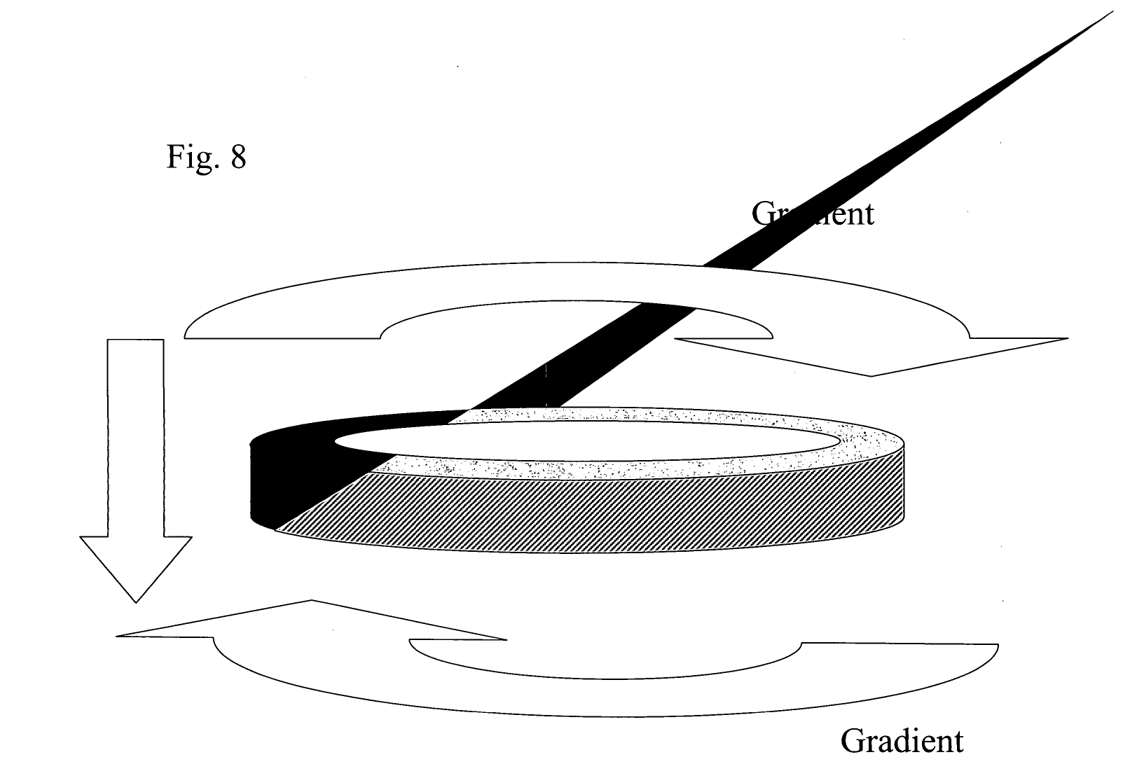

FIG. 8 a microsensor with gradient distribution of the sensor material on the carrier

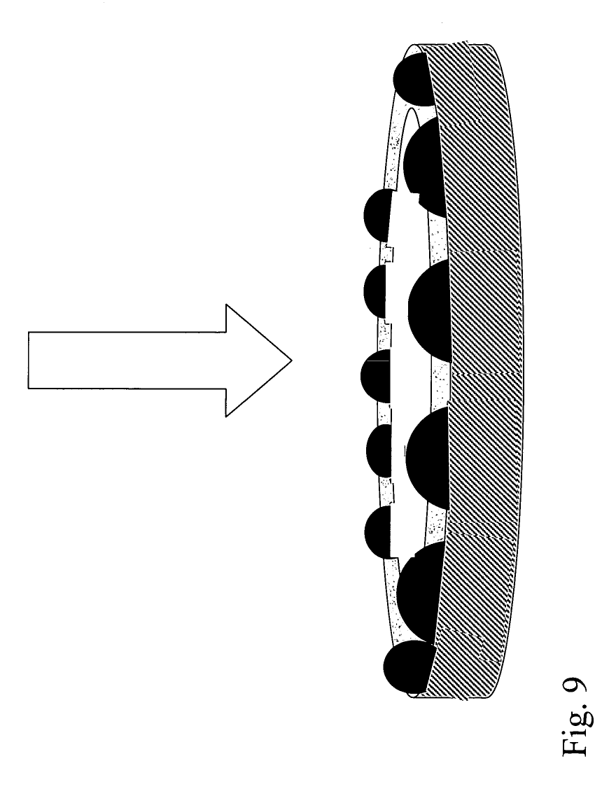

FIG. 9 an example of the occupancy of a ring-shaped sensor device,

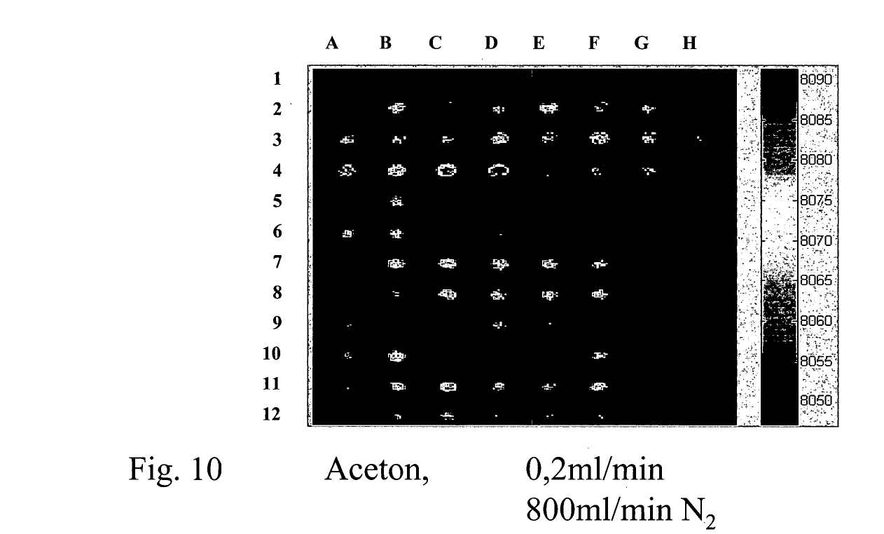

FIG. 10 an infrared picture of acetone,

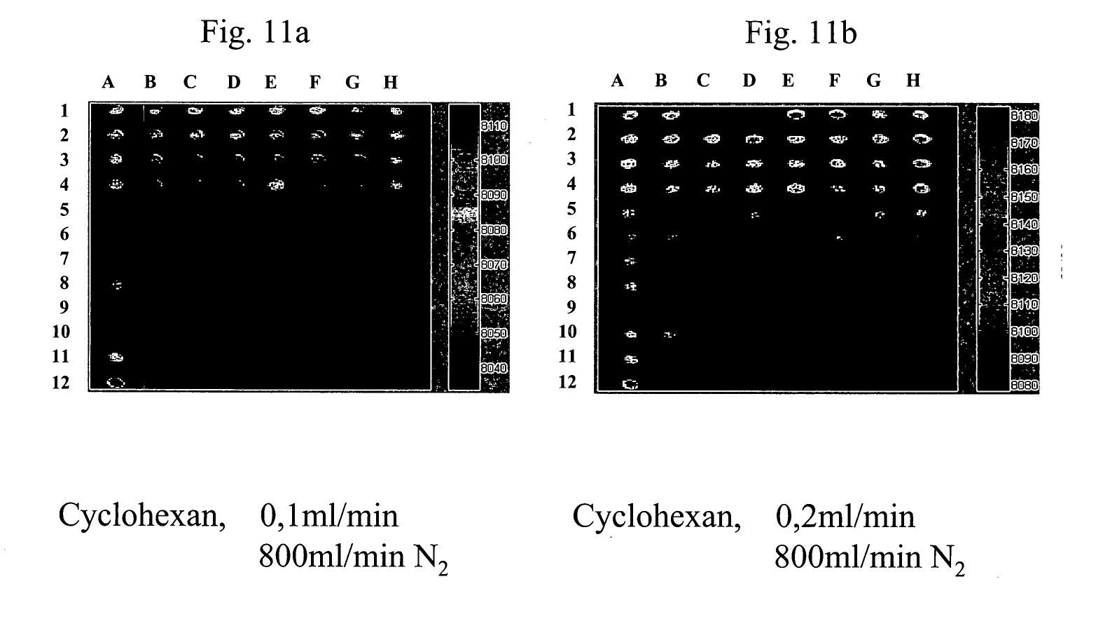

FIGS. 11a, b infrared pictures of cyclohexane with various concentrations,

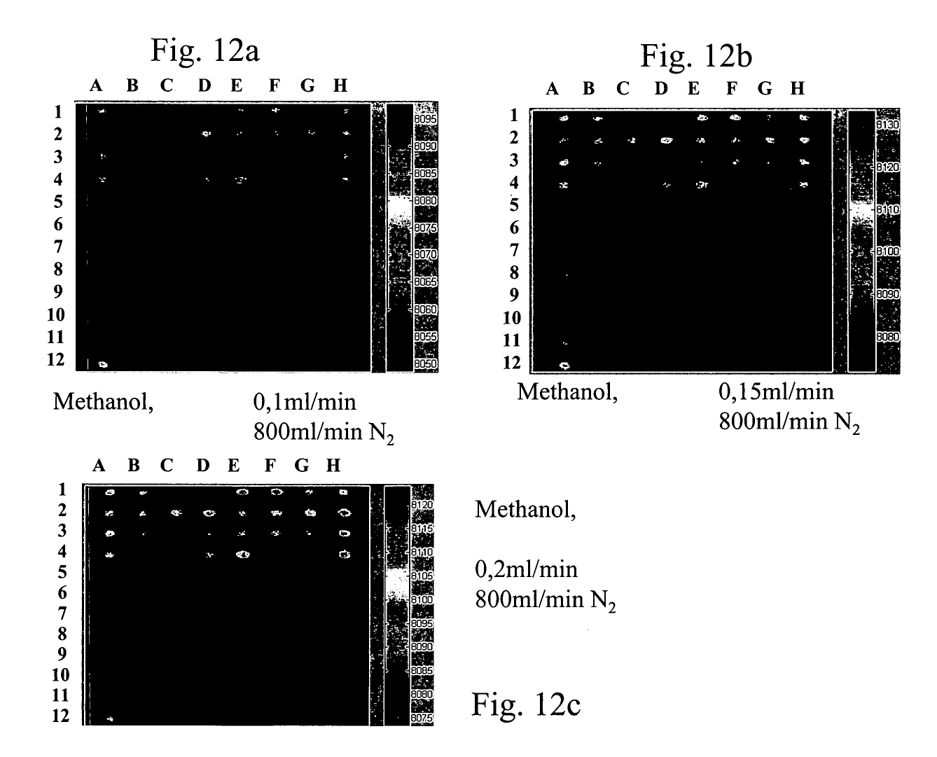

FIGS. 12a, b, c infrared pictures of methanol with various concentrations,

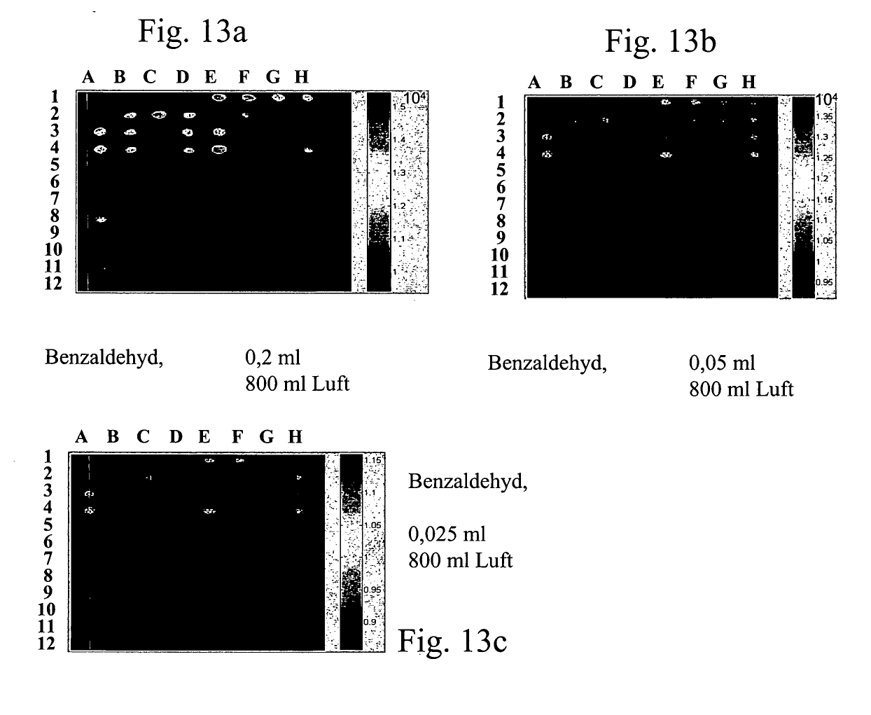

FIGS. 13a, b, c infrared pictures of benzaldehyde with various concentrations; and

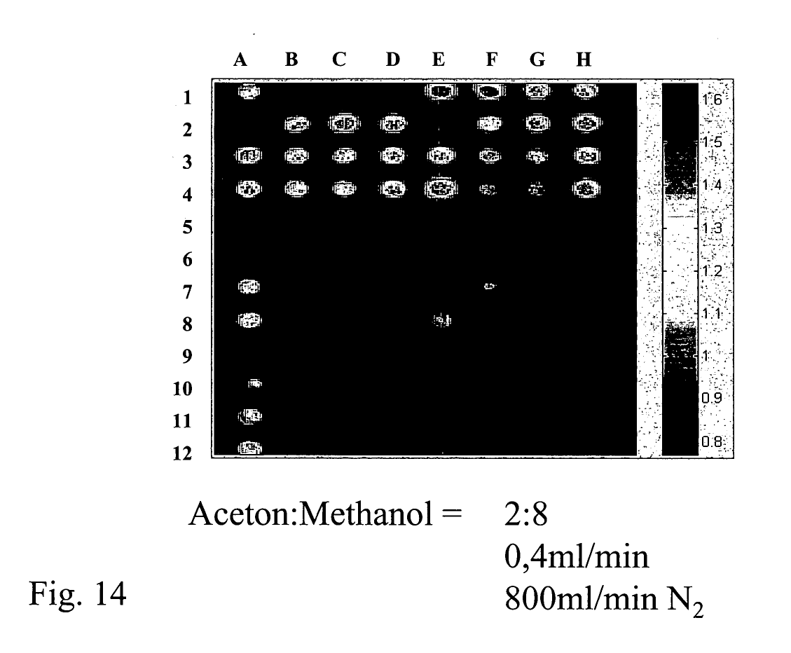

FIG. 14 an infrared picture of an acetone/methanol mixture.

FIG. 1 shows the principle construction of the inventive device 10 with a sensor carrier 12, microsensors 14, an infrared transparent window 16, a heating element 18, a casing 20, a fluid inlet 22, an infrared camera 26 and a data processing facility 30. The arrows in dotted lines under reference number 28 are the field of view of the infrared camera 26.

The infrared camera 26 is preferably connected with the data processing facility 30 via a connection element 32. The connection element 32 may be any of the connection elements known to the person skilled in the art. Preferably, one or more connection cables are used. Alternatively, the connection element may also be an infrared, radio, or other interface without having direct connection, appropriate for the purpose of data transfer.

A preferred embodiment of an array of microsensors 14 in a sensor carrier 12 is shown in FIG. 2. The fluid inlet 22 as well as the fluid outlet 24 are arranged vertically to the section plane in this embodiment and are not shown in this representation.

FIG. 2 shows the example of a 17×9 matrix of microsensors 14. The size, form and arrangement of the matrix may generally be chosen arbitrarily, wherein two or three-dimensional matrixes can be used.

FIG. 3a shows an exchangeable sensor carrier 12 with microsensors 14 in the form of a sandwich unit with infrared transparent window 16 in the cross-section. The sensor carrier 12 as well as the infrared transparent window 16 are, in this embodiment, preferably made of silicon, e.g. as a silicon wafer. Such sandwich units are particularly appropriate as exchangeable units, thus increasing the functionality as well as the flexibility of the device 10 as per the invention. The microsensors 14 are preferably ceramics balls (carriers, preferably coated with one or more sensor materials).

Optionally, as shown in FIG. 3b, a heating element 18 may be preferably provided below the sensor carrier 12. The heating element 18 makes various operating temperatures of the sensor device possible.

FIG. 4a shows a sensor carrier 12, wherein various or equal microsensors 14 are connected in series via channels 34 while they are connected in parallel within the channel. The inlet of the fluid medium to the microsensors 14 is therein performed via the fluid inlet 22 and the discharge via the fluid outlet 24.

The embodiment of FIG. 4a is shown in a cross-sectional view in FIG. 4b for better understanding. FIG. 4b, however, shows only the sensor carrier 12 with the sections 36 provided for receiving the microsensors 14. The connection channels 34 between the sections 36 are clearly recognizable. In contrast to the previous sensor carriers 12, this is a sensor carrier 12 or two parts with an upper part 38 and a lower part 40. Reference 16 again determines the infrared transparent window, which is preferably provided as silicon wafer or sapphire plate. In particular the lower part 40 can be produce of slate for performing the emissivity correction.

FIG. 5 shows a further possibility of a two-dimensional arrangement of an array of equal or various microsensors 14 on a sensor carrier 12 with fluid inlets 22 and fluid outlets 24.

The possibility of a multiport valve circuit in connection with an inventive sensor arrangement is shown in FIG. 6. Therein, various fluid mediums are let in via the fluid inlet 22, controlled/regulated by means of a multiport valve 42 switched in front of the fluid inlet 22. The multiport valve 42 may be an external element, preceding the fluid inlet 22 of the sensor carrier 12, as shown in FIG. 6, or it can be an integral element of the sensor carrier 12.

The combination of the inventive device with a device, e.g. a reactor for testing catalyst arrays, the waste-gas of which is to be analyzed, is shown in FIG. 7.

The inventive device 10 is therein arranged below the test reactor 44, wherein the waste-gas apertures of the test reactor 44 are congruent with the fluid inlet apertures 22 of the inventive device 10. This guarantees, that the waste gas 48 is, at least in this embodiment, is conducted only to one microsensor 14 in the sensor carrier 12, originating from an e.g. catalyst 46 to be tested. Alternatively, the waste-gas flow 48 may also be conducted individually, e.g. in using a multiport valve 42 via the whole sensor device, in particular if various microsensors 14 are provided within an array for analyzing various reaction products of a waste-gas flow.

The sensor carriers 12, described in the previous figures, can also be used instead of or in combination with the sensor carrier device shown in FIG. 7. Generally, the inventive device 10 can be further spaced (spatially separated) from the test reactor 44 wherein appropriate connection elements for conducting the fluid medium to be analyze are then to be provided, which may in turn be heatable.

FIG. 8 shows the principle construction of a microsensor 14 wherein the sensor material is applied onto a ring-shaped carrier in the form of a gradient. The carrier can therein be coated on top or bottom with preferably various concentrations of sensor materials. The ring-shaped carrier of this embodiment is preferably porous.

### EXAMPLE

In the following example, 94 various sensor materials for microsensors, or being microsensors themselves, were synthesized. Table 1 shows the composition of each individual sensor material. All were produced by means of impregnating of Al₂O₃ bodies. Therein, the following aqueous metal salt solutions were used:

FIG. 9 shows the occupancy of the sensor device with the individual microsensors. The positions B1 and E7 contain pure Al₂O₃ materials in order to generate reference positions for no sensor activity whatsoever (blind values).

Within a heatable sensor arrangement (analogous to DE-A 100 12 847.5 and DE-A 101 32 252.6) the microsensors are applied to the positions A1-H12 (=1-96) of a slate plate as per Table 1 and are tempered to 250° C. An IR camera of the type AIM/Aegais PtSi 256×256 is calibrated to the temperature of the sensor device via an emissivity correction (as per WO 99/34206) so that even small differences of temperature in the sensor activity can be detected with high resolution.

A total gas flow of 800 ml/min N₂ flows homogeneously over all microsensors. Via a liquid dosage, various organic fluids are evaporated in the gas flow in the range of 0.05 ml/min to 0.2 ml/min and conducted over the sensor arrangement.

FIG. 10 shows the result of 0.2 ml/min acetone in 800 ml/min N₂. A characteristic pattern is created over the 96 sensor positions A1-H12, wherein the most microsensors on iridium basis show heat emission when contacted with acetone in the rows 1-4. The gray scale values on the right side of FIG. 10 show the intensity of heat toning coded in gray values. It has to be taken into account that the scale ranges from dark to light back to dark, that, e.g., the heat emission on position D4 increases in the middle of the microsensors, and does not decrease.

In contrast therewith, FIGS. 11a and 11b show the analogous experiment with two different concentrations of cyclohexane, wherein FIG. 11b shows the analogous experiment conditions to FIG. 10, merely the organic molecule is different. A completely different intensity and response pattern of the microsensors is clearly recognizable. Cyclohexane causes in the rows 5 to 12 merely sensor activity at the column A as well as the position B10. In FIG. 10 (acetone), large areas are ? (rows 7-12). In the same way, the doubling of the concentration of cyclohexane (FIGS. 11a and 11b) in the gas flow doubles the intensities of the heat emission via corresponding materials.

FIG. 12 shows the results for 3 different methanol concentrations within the gas flow, besides a completely new pattern, the dependency of the intensity of the heat tonings from the concentration of the fluid in the gas phase is recognizable again.

FIG. 13 shows the results when streaming an aromatic aldehyde. Therein, three different concentrations of benzaldehyde are dosed into the gas flow.

FIG. 14 shows the result when streaming an acetone/methanol mixture.

Even the patterns of cyclohexane (FIGS. 11 and 11b) and methanol (FIGS. 12a-c), at first appearing similar, they vary different on the microsensors e.g. at the positions G1, C3, B4, C4, F4, and G4. Even if only one position, i.e. one microsensor or sensor material, differs, a qualitative and quantitative detection of the substance is possible.

After calibration of the arrangement, it is thus easily possible to identify substances in mixtures according to their “pattern” or “fingerprint” on the sensor device. This pattern recognition for qualitative detective can also be automated via a software (comparisons via a large library). For quantitative determination, the device is first calibrated with various concentrations of substances or substance mixtures, the determination of the contents is the performed via the evaluation the intensity of the whole pattern or the intensity of at least one characteristic microsensor or sensor material for this substance or for the mixture.

The following two pages show table 1:

| Pos. | Nr. | Ir | Re | Zn | Pt | Pd | Ru | Rh | Ir | Cu | Ag | Au |
|---|---|---|---|---|---|---|---|---|---|---|---|---|
| A1 | 1 | 3 | 0 | 0 | 3E−04 | 0 | 0 | 0 | 0 | 0 | 0 | 0 |
| B1 | 2 | 0 | 0 | 0 | 0 | 0 | 0 | 0 | 0 | 0 | 0 | 0 |
| C1 | 3 | 3 | 0 | 0 | 0 | 0 | 3E−04 | 0 | 0 | 0 | 0 | 0 |
| D1 | 4 | 3 | 0 | 0 | 0 | 0 | 0 | 3E−04 | 0 | 0 | 0 | 0 |
| E1 | 5 | 3 | 0 | 0 | 0 | 0 | 0 | 0 | 3E−04 | 0 | 0 | 0 |
| F1 | 6 | 3 | 0 | 0 | 0 | 0 | 0 | 0 | 0 | 3E−04 | 0 | 0 |
| G1 | 7 | 3 | 0 | 0 | 0 | 0 | 0 | 0 | 0 | 0 | 3E−04 | 0 |
| H1 | 8 | 3 | 0 | 0 | 0 | 0 | 0 | 0 | 0 | 0 | 0 | 3E−04 |
| A2 | 9 | 2.97 | 0 | 0 | 0.03 | 0 | 0 | 0 | 0 | 0 | 0 | 0 |
| B2 | 10 | 2.97 | 0 | 0 | 0 | 0.03 | 0 | 0 | 0 | 0 | 0 | 0 |
| C2 | 11 | 2.97 | 0 | 0 | 0 | 0 | 0.03 | 0 | 0 | 0 | 0 | 0 |
| D2 | 12 | 2.97 | 0 | 0 | 0 | 0 | 0 | 0.03 | 0 | 0 | 0 | 0 |
| E2 | 13 | 2.97 | 0 | 0 | 0 | 0 | 0 | 0 | 0.03 | 0 | 0 | 0 |
| F2 | 14 | 2.97 | 0 | 0 | 0 | 0 | 0 | 0 | 0 | 0.03 | 0 | 0 |
| G2 | 15 | 2.97 | 0 | 0 | 0 | 0 | 0 | 0 | 0 | 0 | 0.03 | 0 |
| H2 | 16 | 2.97 | 0 | 0 | 0 | 0 | 0 | 0 | 0 | 0 | 0 | 0.03 |
| A3 | 17 | 2.7 | 0 | 0 | 0.3 | 0 | 0 | 0 | 0 | 0 | 0 | 0 |
| B3 | 18 | 2.7 | 0 | 0 | 0 | 0.3 | 0 | 0 | 0 | 0 | 0 | 0 |
| C3 | 19 | 2.7 | 0 | 0 | 0 | 0 | 0.3 | 0 | 0 | 0 | 0 | 0 |
| D3 | 20 | 2.7 | 0 | 0 | 0 | 0 | 0 | 0.3 | 0 | 0 | 0 | 0 |
| E3 | 21 | 2.7 | 0 | 0 | 0 | 0 | 0 | 0 | 0.3 | 0 | 0 | 0 |
| F3 | 22 | 2.7 | 0 | 0 | 0 | 0 | 0 | 0 | 0 | 0.3 | 0 | 0 |
| G3 | 23 | 2.7 | 0 | 0 | 0 | 0 | 0 | 0 | 0 | 0 | 0.3 | 0 |
| H3 | 24 | 2.7 | 0 | 0 | 0 | 0 | 0 | 0 | 0 | 0 | 0 | 0.3 |
| A4 | 25 | 2.5 | 0 | 0 | 0.5 | 0 | 0 | 0 | 0 | 0 | 0 | 0 |
| B4 | 26 | 2.5 | 0 | 0 | 0 | 0.5 | 0 | 0 | 0 | 0 | 0 | 0 |
| C4 | 27 | 2.5 | 0 | 0 | 0 | 0 | 0.5 | 0 | 0 | 0 | 0 | 0 |
| D4 | 28 | 2.5 | 0 | 0 | 0 | 0 | 0 | 0.5 | 0 | 0 | 0 | 0 |
| E4 | 29 | 2.5 | 0 | 0 | 0 | 0 | 0 | 0 | 0.5 | 0 | 0 | 0 |
| F4 | 30 | 2.5 | 0 | 0 | 0 | 0 | 0 | 0 | 0 | 0.5 | 0 | 0 |
| G4 | 31 | 2.5 | 0 | 0 | 0 | 0 | 0 | 0 | 0 | 0 | 0.5 | 0 |
| H4 | 32 | 2.5 | 0 | 0 | 0 | 0 | 0 | 0 | 0 | 0 | 0 | 0.5 |
| A5 | 33 | 0 | 3 | 0 | 3E−04 | 0 | 0 | 0 | 0 | 0 | 0 | 0 |
| B5 | 34 | 0 | 3 | 0 | 0 | 3E−04 | 0 | 0 | 0 | 0 | 0 | 0 |
| C5 | 35 | 0 | 3 | 0 | 0 | 0 | 3E−04 | 0 | 0 | 0 | 0 | 0 |
| D5 | 36 | 0 | 3 | 0 | 0 | 0 | 0 | 3E−04 | 0 | 0 | 0 | 0 |
| E5 | 37 | 0 | 3 | 0 | 0 | 0 | 0 | 0 | 3E−04 | 0 | 0 | 0 |
| F5 | 38 | 0 | 3 | 0 | 0 | 0 | 0 | 0 | 0 | 3E−04 | 0 | 0 |
| G5 | 39 | 0 | 3 | 0 | 0 | 0 | 0 | 0 | 0 | 0 | 3E−04 | 0 |
| H5 | 40 | 0 | 3 | 0 | 0 | 0 | 0 | 0 | 0 | 0 | 0 | 3E−04 |
| A6 | 41 | 0 | 2.97 | 0 | 0.03 | 0 | 0 | 0 | 0 | 0 | 0 | 0 |
| B6 | 42 | 0 | 2.97 | 0 | 0 | 0.03 | 0 | 0 | 0 | 0 | 0 | 0 |
| C6 | 43 | 0 | 2.97 | 0 | 0 | 0 | 0.03 | 0 | 0 | 0 | 0 | 0 |
| D6 | 44 | 0 | 2.97 | 0 | 0 | 0 | 0 | 0.03 | 0 | 0 | 0 | 0 |
| E6 | 45 | 0 | 2.97 | 0 | 0 | 0 | 0 | 0 | 0.03 | 0 | 0 | 0 |
| F6 | 46 | 0 | 2.97 | 0 | 0 | 0 | 0 | 0 | 0 | 0.03 | 0 | 0 |
| G6 | 47 | 0 | 2.97 | 0 | 0 | 0 | 0 | 0 | 0 | 0 | 0.03 | 0 |
| H6 | 48 | 0 | 2.97 | 0 | 0 | 0 | 0 | 0 | 0 | 0 | 0 | 0.03 |
| A7 | 49 | 0 | 2.7 | 0 | 0.3 | 0 | 0 | 0 | 0 | 0 | 0 | 0 |
| B7 | 50 | 0 | 2.7 | 0 | 0 | 0.3 | 0 | 0 | 0 | 0 | 0 | 0 |
| C7 | 51 | 0 | 2.7 | 0 | 0 | 0 | 0.3 | 0 | 0 | 0 | 0 | 0 |
| D7 | 52 | 0 | 2.7 | 0 | 0 | 0 | 0 | 0.3 | 0 | 0 | 0 | 0 |
| E7 | 53 | 0 | 0 | 0 | 0 | 0 | 0 | 0 | 0 | 0 | 0 | 0 |
| F7 | 54 | 0 | 2.7 | 0 | 0 | 0 | 0 | 0 | 0 | 0.3 | 0 | 0 |
| G7 | 55 | 0 | 2.7 | 0 | 0 | 0 | 0 | 0 | 0 | 0 | 0.3 | 0 |
| H7 | 56 | 0 | 2.7 | 0 | 0 | 0 | 0 | 0 | 0 | 0 | 0 | 0.3 |
| A8 | 57 | 0 | 2.5 | 0 | 0.5 | 0 | 0 | 0 | 0 | 0 | 0 | 0 |
| B8 | 58 | 0 | 2.5 | 0 | 0 | 0.5 | 0 | 0 | 0 | 0 | 0 | 0 |
| C8 | 59 | 0 | 2.5 | 0 | 0 | 0 | 0.5 | 0 | 0 | 0 | 0 | 0 |
| D8 | 60 | 0 | 2.5 | 0 | 0 | 0 | 0 | 0.5 | 0 | 0 | 0 | 0 |
| E8 | 61 | 0 | 2.5 | 0 | 0 | 0 | 0 | 0 | 0.5 | 0 | 0 | 0 |
| F8 | 62 | 0 | 2.5 | 0 | 0 | 0 | 0 | 0 | 0 | 0.5 | 0 | 0 |
| G8 | 63 | 0 | 2.5 | 0 | 0 | 0 | 0 | 0 | 0 | 0 | 0.5 | 0 |
| H8 | 64 | 0 | 2.5 | 0 | 0 | 0 | 0 | 0 | 0 | 0 | 0 | 0.5 |
| A9 | 65 | 0 | 0 | 3 | 3E−04 | 0 | 0 | 0 | 0 | 0 | 0 | 0 |
| B9 | 66 | 0 | 0 | 3 | 0 | 3E−04 | 0 | 0 | 0 | 0 | 0 | 0 |
| C9 | 67 | 0 | 0 | 3 | 0 | 0 | 3E−04 | 0 | 0 | 0 | 0 | 0 |
| D9 | 68 | 0 | 0 | 3 | 0 | 0 | 0 | 3E−04 | 0 | 0 | 0 | 0 |
| E9 | 69 | 0 | 0 | 3 | 0 | 0 | 0 | 0 | 3E−04 | 0 | 0 | 0 |
| F9 | 70 | 0 | 0 | 3 | 0 | 0 | 0 | 0 | 0 | 3E−04 | 0 | 0 |
| G9 | 71 | 0 | 0 | 3 | 0 | 0 | 0 | 0 | 0 | 0 | 3E−04 | 0 |
| H9 | 72 | 0 | 0 | 3 | 0 | 0 | 0 | 0 | 0 | 0 | 0 | 3E−04 |
| A10 | 73 | 0 | 0 | 2.97 | 0.03 | 0 | 0 | 0 | 0 | 0 | 0 | 0 |
| B10 | 74 | 0 | 0 | 2.97 | 0 | 0.03 | 0 | 0 | 0 | 0 | 0 | 0 |
| C10 | 75 | 0 | 0 | 2.97 | 0 | 0 | 0.03 | 0 | 0 | 0 | 0 | 0 |
| D10 | 76 | 0 | 0 | 2.97 | 0 | 0 | 0 | 0.03 | 0 | 0 | 0 | 0 |
| E10 | 77 | 0 | 0 | 2.97 | 0 | 0 | 0 | 0 | 0.03 | 0 | 0 | 0 |
| F10 | 78 | 0 | 0 | 2.97 | 0 | 0 | 0 | 0 | 0 | 0.03 | 0 | 0 |
| G10 | 79 | 0 | 0 | 2.97 | 0 | 0 | 0 | 0 | 0 | 0 | 0.03 | 0 |
| H10 | 80 | 0 | 0 | 2.97 | 0 | 0 | 0 | 0 | 0 | 0 | 0 | 0.03 |
| A11 | 81 | 0 | 0 | 2.7 | 0.3 | 0 | 0 | 0 | 0 | 0 | 0 | 0 |
| B11 | 82 | 0 | 0 | 2.7 | 0 | 0.3 | 0 | 0 | 0 | 0 | 0 | 0 |
| C11 | 83 | 0 | 0 | 2.7 | 0 | 0 | 0.3 | 0 | 0 | 0 | 0 | 0 |
| D11 | 84 | 0 | 0 | 2.7 | 0 | 0 | 0 | 0.3 | 0 | 0 | 0 | 0 |
| E11 | 85 | 0 | 0 | 2.7 | 0 | 0 | 0 | 0 | 0.3 | 0 | 0 | 0 |
| F11 | 86 | 0 | 0 | 2.7 | 0 | 0 | 0 | 0 | 0 | 0.3 | 0 | 0 |
| G11 | 87 | 0 | 0 | 2.7 | 0 | 0 | 0 | 0 | 0 | 0 | 0.3 | 0 |
| H11 | 88 | 0 | 0 | 2.7 | 0 | 0 | 0 | 0 | 0 | 0 | 0 | 0.3 |
| A12 | 89 | 0 | 0 | 2.5 | 0.5 | 0 | 0 | 0 | 0 | 0 | 0 | 0 |
| B12 | 90 | 0 | 0 | 2.5 | 0 | 0.5 | 0 | 0 | 0 | 0 | 0 | 0 |
| C12 | 91 | 0 | 0 | 2.5 | 0 | 0 | 0.5 | 0 | 0 | 0 | 0 | 0 |
| D12 | 92 | 0 | 0 | 2.5 | 0 | 0 | 0 | 0.5 | 0 | 0 | 0 | 0 |
| E12 | 93 | 0 | 0 | 2.5 | 0 | 0 | 0 | 0 | 0.5 | 0 | 0 | 0 |
| F12 | 94 | 0 | 0 | 2.5 | 0 | 0 | 0 | 0 | 0 | 0.5 | 0 | 0 |
| G12 | 95 | 0 | 0 | 0 | 0 | 0 | 0 | 0 | 0 | 0 | 0 | 0 |
| H12 | 96 | 0 | 0 | 2.5 | 0 | 0 | 0 | 0 | 0 | 0 | 0 | 0.5 |

List of References:

10—device in accordance with the invention

12—sensor carrier

14—micro sensor

16—infrared transparent window

18—heating element

20—casing

22—fluid inlet

24—fluid outlet

26—infrared camera

28—field of view of infrared camera

30—data processing facility

32—connection element

34—channel

36—section

38—upper part

40—lower part

42—multiport valve

44—test reactor

46—catalyst

48—waste-gas direction

1. Method for the analysis of a fluid medium wherein the fluid medium is guided over at least one micro sensor (14) of a sensor assembly, comprising at least two microsensors (14) being identical or different, and wherein the at least one microsensor (14) is thermographically monitored with at least one detector with regard to a change of properties, wherein the change of properties of the at least one microsensor (14) is specific for at least one predetermined component of the fluid medium.

2. Method in accordance with claim 1 wherein the signal transfer from the at least one microsensor (14) to the at least one detector is performed without any electrically conducting connection, in particular wireless.

3. Method in accordance with claim 1 or 2, wherein the at least one detector is an infrared camera (26) or an assembly of infrared diodes or a traverseable infrared diode.

4. Method in accordance with claim 1 wherein the change of properties is chosen from: thermoelectric effect, Peltier effect, Chelat effect, adsorption such as chemisorption and physisorption, desoprtion, catalytic reactions, formation of complexes, molecular recognition.

5. Use of the method in accordance with claim 1 for determination of performance properties of building blocks of material libraries.

6. Use of the method in accordance with claim 1 for detection of drugs, samples, explosives, combat means, pesticides, environmental poisons; for applications in the process control, quality control in the food, animal food and cosmetics industries, in the field of forensics (forensic detection), in the securing of evidence; for chemical analysis with or without coupling with already-known analyzing methods; for olfactory tests in the consumer goods industry; for detecting mineral resources; in the extra-terrestrial research.

7. Device comprising:
(1) means for receiving at least two individual microsensors (14) with or without a sensor carrier (12) with at least two different sections (36), separated from each other, for providing a sensor assembly,
(2) means for guiding in and out of at least one fluid medium into at least one section (36) of the sensor device with simultaneous contacting of the fluid medium with at least one microsensor (14),
(3) at least one detector for thermographic determination of at least one amendment of properties of the microsensor (14), as well as
(4) a data processing facility (30) which processes the sections (36) detected when measuring the at least one change of properties with the corresponding measuring values according to their position within the sensor carrier (12).

8. Device (10) in accordance with claim 7 characterized in that the means for receiving the at least two individual microsensors (14) and/or the at least two individual microsensors (14) are arbitrarily exchangeable.

9. Device (10) in accordance with claim 7, furthermore comprising a casing (20) wherein the sensor carrier (12) is provided.

10. Device (10) in accordance with claim 7, wherein the device (10) comprises means for heating (18) and/or means for refrigerating of the casing (20) and/or the sensor carrier (12).

11. Device (10) in accordance with claim 9, characterized in that the casing (20) comprises an infrared transparent window (16) and that the infrared detector is provided outside the casing (20) in front of the infrared transparent window (16).

12. Device (10) in accordance with claim 9 characterized in that the casing (20) and/or the sensor carrier (12) consist completely or partially of silicon and/or sapphire or another appropriate material, transparent for infrared, or slate.

13. Device in accordance with claim 9 characterized in that the means for guiding in and out of the at least one fluid medium comprise channels (34) within the sensor carrier (12).

14. Device (10) in accordance with claim 13, characterized in that the channels (34) connect more than one sections with one ? (36).

15. Device (10) in accordance with claim 9 further comprising at least one multiport valve (42) and/or a device for equal distribution of the fluid medium over at least one section of the sensor assembly.

16. Sensor assembly comprising at least two microsensors (14) wherein the composition of the microsensors (14) changes continuously or discontinuously.

17. Use of the microsensor (14) or the method or the device (10) in accordance with one of the preceding claims for controlling chemical conversions.

18. Computer program with program code means for performing the method in accordance with claim 1 or for controlling and/or regulating the device (10) in accordance with claim 7 to 15.

19. Data carrier with computer program in accordance with claim 18.

20. Method in accordance with claim 1 wherein the evaluation is effected in accordance with a recognition of pattern in such a way that the detected measuring values are compared with pattern data banks and that, thereof, preferably automatically, identity and quantity of the at least one component in the fluid medium is determined.

21. Use of the method in accordance with claim 1 as well as 20 for developing and localization of new sensor materials.
